(19) United States
(12) Patent Application Publication
(10) Pub. No.: US 2021/0230640 A1
(43) Pub. Date: Jul. 29, 2021

(54) METHODS FOR CONTROLLING FERMENTATION FEED RATES

(71) Applicant: AMYRIS, INC., Emeryville, CA (US)

(72) Inventor: Joshua LENG, Emeryville, CA (US)

(21) Appl. No.: 17/255,348

(22) PCT Filed: Jul. 11, 2019
(86) PCT No.: PCT/US2019/041452

§ 371 Date: Dec. 22, 2020

Related U.S. Application Data
(60) Provisional application No. 62/697,167, filed on Jul. 12, 2018.

(51) Int. Cl. C12P 5/00; C12P 7/64; C12P 7/24

(52) U.S. Cl. CPC C12P 5/007; C12P 7/24; C12P 7/6409

(57) ABSTRACT

The present disclosure relates to methods for enhancing productivity or yield in fermentation. The methods provide monitoring one or more compounds produced by culture cells as a feedback control for the feed rate of the fermentation.

### 1. TECHNICAL FIELD

The present disclosure relates to the use of off-gas monitoring to control or adjust the feed rate of a fermentation medium and apparatuses for the same.

### 2. BACKGROUND OF THE INVENTION

The advent of synthetic biology has brought about the promise of fermentative microbial production of biofuels, chemicals and biomaterials from renewable sources at industrial scale and quality. For example, functional non-native biological pathways have been successfully constructed in microbial hosts for the production of precursors to the antimalarial drug artemisinin (see, e.g., Martin et al., Nat Biotechnol 21:796-802 (2003); fatty acid derived fuels and chemicals (e.g., fatty esters, fatty alcohols and waxes; see, e.g., Steen et al., Nature 463:559-562 (2010); polyketide synthases that make cholesterol lowering drugs (see, e.g., Ma et al., Science 326:589-592 (2009); and polyketides (see, e.g., Kodumal, Proc Natl Acad Sci USA 101:15573-15578 (2004). However, the commercial success of synthetic biology will depend largely on whether the production cost of renewable products can be made to compete with, or out-compete, the production costs of their respective non-renewable counterparts

Some of the largest costs of synthetic biology occur during fermentation. Much of these costs go toward the ingredients of the fermentation media including carbon sources, nitrogen sources, water, salts, and nutrients. In a conventional fermentation, a pulse of sugar is fed to a culture until a spike in dissolved oxygen is detected, indicating consumption of excess carbon. Then, a new pulse of sugar is fed to the culture to start a new cycle. These cycles of pulse feeding and checking for dissolved oxygen spikes repeat throughout the fermentation, resulting in inefficient consumption of feed and labor for the practitioner. Methods and compositions that improve the yields of fermentations will reduce the overall costs, making the production of renewable compounds more efficient and competitive.

### 3. SUMMARY OF THE INVENTION

Provided herein are processes for providing feed to microbial cultures in a fermentation medium. In some embodiments, feed is provided to a microbial culture in a fermentation medium at an initial rate. The concentration of a volatile cell product is measured in the off-gas from the fermentation medium. In some embodiments, when the concentration of the volatile cell product increases, the feed rate is decreased; and, when the concentration of the volatile cell product decreases, the feed rate is increased. In some embodiments, when the concentration of the volatile cell product decreases, the feed rate is decreased; and, when the concentration of the volatile cell product increases, the feed rate is increased. Advantageously, in particular embodiments, the volatile cell product can be measured rapidly or continuously, and the feed rate can be adjusted rapidly or continuously. These method steps can be carried out with techniques and components apparent to those of skill in the art. Particular techniques and components are described in detail herein.

As described in detail below, the methods and compositions provided herein can increase productivity of a microbial strain by up to 15%, or more. An increase of 15% in productivity provides a direct improvement in costs and efficiency for such a fermentation.

### 4. BRIEF DESCRIPTION OF FIGURES

FIG. 1 provides a history plot of feed rate (g/L/hr), ethanol (ppm), temperature (° C.), pH, volume (L), oxygen uptake rate (mM/L-min), stir rate (rpm), pO₂ (% saturation), air flow (slpm), and average pO₂ (% saturation).

FIG. 2 provides productivity (g/L/hr) as a function of average off-gas ethanol concentration (ppm) for two different strains.

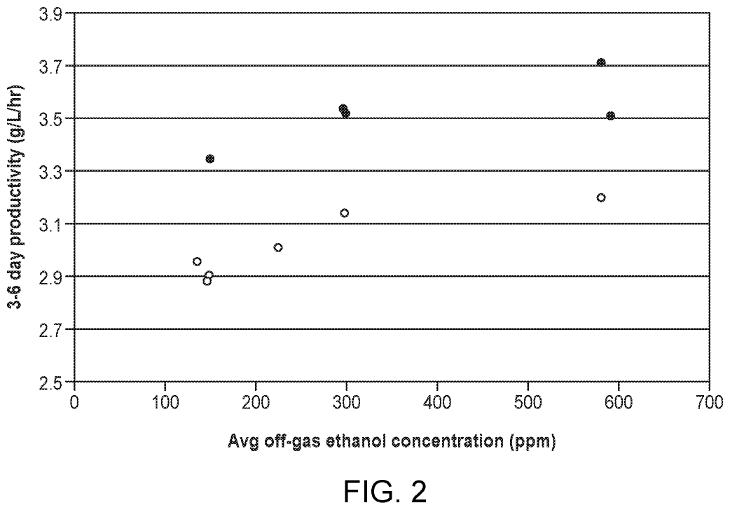

FIG. 3 provides ethanol off-gas concentrations for fermentations of a yeast strain according to the methods provided herein.

### 5. DESCRIPTION OF EMBODIMENTS

### 5.1 Definitions

As used herein, the term “genetically modified” refers to a host cell that comprises a heterologous nucleotide sequence.

As used herein, the term “heterologous” refers to what is not normally found in nature. The term “heterologous nucleotide sequence” refers to a nucleotide sequence not normally found in a given cell in nature. As such, a heterologous nucleotide sequence may be: (a) foreign to its host cell (i.e., is “exogenous” to the cell); (b) naturally found in the host cell (i.e., “endogenous”) but present at an unnatural quantity in the cell (i.e., greater or lesser quantity than naturally found in the host cell); or (c) be naturally found in the host cell but positioned outside of its natural locus. The term “heterologous enzyme” refers to an enzyme that is not normally found in a given cell in nature. The term encompasses an enzyme that is: (a) exogenous to a given cell (i.e., encoded by a nucleotide sequence that is not naturally present in the host cell or not naturally present in a given context in the host cell); and (b) naturally found in the host cell (e.g., the enzyme is encoded by a nucleotide sequence that is endogenous to the cell) but that is produced in an unnatural amount (e.g., greater or lesser than that naturally found) in the host cell.

On the other hand, the term “native” or “endogenous” as used herein with reference to molecules, and in particular enzymes and nucleic acids, indicates molecules that are expressed in the organism in which they originated or are found in nature, independently of the level of expression that can be lower, equal, or higher than the level of expression of the molecule in the native microorganism. It is understood that expression of native enzymes or polynucleotides may be modified in recombinant microorganisms.

As used herein, the term “production” generally refers to an amount of a compound produced by a host cell provided herein. In some embodiments, production is expressed as a yield of compound by the host cell. In other embodiments, production is expressed as a productivity of the host cell in producing the compound.

As used herein, the term “productivity” refers to production of a compound by a host cell, expressed as the amount of compound produced (by weight) per amount of fermentation broth in which the host cell is cultured (by volume) over time (per hour).

As used herein, the term “yield” refers to production of a compound by a host cell, expressed as the amount of compound produced per amount of carbon source consumed by the host cell, by weight.

### 5.2 Description

In one aspect, provided herein are methods for providing a feed compound to a microbial culture. In certain embodiments, the methods comprise the following steps: feeding a feed compound to a microbial culture growing in fermentation medium at an initial rate; measuring the concentration of a volatile cell product in off-gas from the fermentation medium; and adjusting the rate of feeding the feed compound to the fermentation medium. In certain embodiments, when the concentration of the volatile cell product increases, the feed rate is decreased; and, when the concentration of the volatile cell product decreases, the feed rate is increased. In some embodiments, when the concentration of the volatile cell product decreases, the feed rate is decreased; and, when the concentration of the volatile cell product increases, the feed rate is increased. Advantageously, in particular embodiments, the volatile cell product can be measured rapidly or continuously, and the feed rate can be adjusted rapidly or continuously. In certain embodiments, the volatile cell product is measured continuously, and the feed rate is adjusted continuously.

The feed compound can be any compound deemed useful by a practitioner for feeding a fermentation medium. In certain embodiments, the feed compound is selected from carbon sources, nitrogen sources, salts, nutrients, and combinations thereof. In particular embodiments, the feed compound is a carbon source. In certain embodiments, the feed compound is provided by a source selected from the group consisting of molasses, corn steep liquor, sugar cane juice, and sugar beet juice. In certain embodiments, the feed compound is a sugar. In some embodiments, the feed compound is a monosaccharide (simple sugar), a disaccharide, a polysaccharide, a non-fermentable carbon source, or one or more combinations thereof. Non-limiting examples of suitable monosaccharides include glucose, galactose, mannose, fructose, xylose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The volatile cell product can be any off-gas compound produced by the microbial culture deemed suitable by the practitioner. In particular embodiments, the volatile cell product is a compound that indicates the metabolic state of the cell that produced it. In certain embodiments, ethanol is measured in the off-gas. In particular embodiments, when the ethanol concentration in the off-gas increases, the feed rate is decreased, and when the ethanol concentration in the off-gas decreases, the feed rate is increased. In certain embodiments, ethanol is measured in the off-gas, and the feed compound is a sugar. In particular embodiments, when the ethanol concentration in the off-gas increases, the sugar feed rate is decreased, and when the ethanol concentration in the off-gas decreases, the sugar feed rate is increased.

The methods provided herein take advantage of the observation that ethanol in the fermentation can be a facile marker for the feed consumption by the cells in the microbial culture. Advantageously, ethanol in the off-gas can be measured routinely by standard techniques and components. According to the discovery provided herein, by adjusting feed rate to maintain a constant or near-constant ethanol concentration in the off-gas, the microbial culture can grow at a more optimal rate compared to standard techniques. This results in improved efficiency, yield, and productivity. As shown in the examples below, quantitative improvements in yield have been achieved with the methods provided herein.

In certain embodiments, the feed rate is maintained within a range of the initial feed rate. The range can be any range deemed suitable by the practitioner of skill. In certain embodiments, the feed rate is maintained within ±75%, ±50%, ±25%, ±15%, ±10%, or ±5% of the initial feed rate. The feed rate can be adjusted according to standard techniques. In certain embodiments, the feed rate is maintained within 0.01 to 25 g/L/hr. In certain embodiments, the feed rate is maintained within 0.1 to 25 g/L/hr. In certain embodiments, the feed rate is maintained within 1 to 25 g/L/hr. The feed rate is typically expressed as mass of feed compound(s) per liter of fermentation medium per unit time. In typical embodiments, the flow of a feed solution in to the fermentation medium is increased or decreased in order to increase or decrease the feed rate.

In certain embodiments, the oxygen uptake rate of the microbial culture is maintained within a range of the initial oxygen uptake rate. The range can be any range deemed suitable by the practitioner of skill. In certain embodiments, the oxygen uptake rate is maintained within ±75%, ±50%, ±25%, ±15%, ±10%, or ±5% of the oxygen uptake rate. In certain embodiments, oxygen uptake rate of the microbial culture is maintained within 1-150 mmol O₂/L/hr. In certain embodiments, oxygen uptake rate of the microbial culture is maintained within 10-150 mmol O₂/L/hr. In certain embodiments, oxygen uptake rate of the microbial culture is maintained within 25-150 mmol O₂/L/hr. Oxygen uptake rate can be maintained by standard techniques, for instance sparging and/or agitation.

In certain embodiments, the volatile cell product in the off-gas is maintained within a range. The range can be any range deemed suitable by the practitioner of skill. In certain embodiments, the volatile cell product is maintained within ±75%, ±50%, ±25%, ±15%, ±10%, or ±5% of a target amount. In certain embodiments, the volatile cell product is ethanol, and the ethanol in the off-gas is maintained at about 600 ppm. In certain embodiments, ethanol in the off-gas is maintained at 50-750 ppm. In certain embodiments, ethanol in the off-gas is maintained at 100-200 ppm. In certain embodiments, ethanol in the off-gas is maintained at 200-300 ppm. In certain embodiments, ethanol in the off-gas is maintained at 250-350 ppm. In certain embodiments, ethanol in the off-gas is maintained at 550-650 ppm. In certain embodiments, the target ethanol concentration in the off-gas is selected from 100 ppm, 150 ppm, 200 ppm, 250 ppm, 300 ppm, 350 ppm, 400 ppm, 450 ppm, 500 ppm, 550 ppm, 600 ppm, 650 ppm, 700 ppm, and 750 ppm.

The amount of the volatile cell product should be maintained according to the techniques described herein. In other words, when the amount of the volatile cell product deviates from the target value, the feed rate of the fermentation should be adjusted. In certain embodiments, when the amount of the volatile cell product increases, the feed rate should be decreased, and when the amount of the volatile cell product decreases, the feed rate should be increased. In other embodiments, when the amount of the volatile cell product increases, the feed rate should be increased, and when the amount of the volatile cell product decreases, the feed rate should be decreased. In particular embodiments, the volatile cell product is ethanol. In these embodiments, when the amount of ethanol in the off-gas increases, the feed rate should be decreased, and when the amount of ethanol in the off-gas decreases, the feed rate should be increased.

The off-gas can be measured at any frequency deemed suitable by the practitioner. In advantageous embodiments, the off-gas is measured at a high frequency. In particular embodiments, the off-gas is measured continuously. In certain embodiments, the off-gas is measured at least once per hour, at least once per half hour, at least once per quarter hour, at least once per 10 minutes, at least once per five minutes, at least once per minute, at least twice per minute, at least three times per minute, at least four times per minute, or at least ten times per minute. In preferred embodiments, the off-gas is measured continuously.

The feed rate can be adjusted at any frequency deemed suitable by the practitioner. In advantageous embodiments, the feed rate is adjusted at a high frequency. In particular embodiments, the feed rate is adjusted continuously. Typically, the feed rate is adjusted following measurement of the volatile cell product in the off-gas. In certain embodiments, the feed rate is adjusted at least once per hour, at least once per half hour, at least once per quarter hour, at least once per 10 minutes, at least once per five minutes, at least once per minute, at least twice per minute, at least three times per minute, at least four times per minute, or at least ten times per minute. In preferred embodiments, the feed rate is adjusted continuously.

The methods provided herein have provided improvements in productivity and yield for fermentations of microbial cultures, for instance microbial cultures producing a compound of interest. In certain embodiments, productivity is increased 5%, 10%, 15%, 20%, 25%, 30%, 35%, 40%, 45%, 50%, or more. In certain embodiments, yield is increased 5%, 10%, 15%, 20%, 25%, 30%, 35%, 40%, 45%, 50%, or more. In certain embodiments, productivity and yield are increased. In these embodiments, productivity or yield increases are relative to the same cell strain grown under conventional conditions, i.e. without the processes described herein.

The methods described herein are cycled several times throughout a fermentation. In some embodiments, the methods described herein are carried out for a period of between 3 and 20 days. In some embodiments, the methods described herein are carried out fora period of 1, 2, 3, 4, 5, 6, 7, 8, 9, 10, 11, 12, 13, 14, 15, 16, 17, 18, 19, 20 or more than 20 days.

### 5.3 Cell Cultures, Media, and Conditions

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the cell culture, the fermentation, and the process.

The methods provided herein may be performed in a suitable culture medium (e.g., with or without pantothenate supplementation) in a suitable container, including but not limited to a cell culture plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, Volume 12, pages 398-473, Wiley-VCH Verlag GmbH & Co. KDaA, Weinheim, Germany.

In some embodiments, the culture medium is any culture medium in which a cell culture can subsist, i.e., maintain growth and viability. In some embodiments, the culture medium is an aqueous medium comprising assimilable carbon, nitrogen and phosphate sources. Such a medium can also include appropriate salts, minerals, metals and other nutrients.

Suitable conditions and suitable media for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

The concentration of a carbon source, such as glucose, in the culture medium should promote cell growth, but not be so high as to repress growth of the microorganism used. In preferred embodiments, the carbon source is at undetectable levels in the fermentation medium (e.g. at less than about 0.1 g/L). In such embodiments, the culture is carbon-limited, and culture cells should consume the carbon source as soon as it is delivered. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals, or growth promoters. Such other compounds can also be present in carbon, nitrogen or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate and mixtures thereof. Typically, the concentration of phosphate in the culture medium is greater than about 1.0 g/L, preferably greater than about 2.0 g/L and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 ml/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture media can include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or isoprenoid production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, a small amount of anti-foaming agent may be added during the culture.

The temperature of the culture medium can be any temperature suitable for growth of the genetically modified cells and/or production of isoprenoid. For example, prior to inoculation of the culture medium with an inoculum, the culture medium can be brought to and maintained at a temperature in the range of from about 20° C. to about 45° C., preferably to a temperature in the range of from about 25° C. to about 40° C., and more preferably in the range of from about 28° C. to about 32° C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

In some embodiments, the carbon source concentration, such as the glucose concentration, of the culture medium is monitored during culture. Glucose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. As described elsewhere, the feed rate of the carbon source is adjusted according to the methods provided herein. The use of aliquots of the original culture medium may be desirable because the concentrations of certain nutrients in the medium (e.g. the nitrogen and phosphate sources) can be maintained simultaneously. Likewise, the trace metals concentrations can be maintained in the culture medium by addition of aliquots of the trace metals solution.

### 5.4 Cells

The cell cultures can comprise any cells deemed useful by those of skill. Cells useful in the compositions and methods provided herein include archae, prokaryotic, or eukaryotic cells. In certain embodiments, the cells are recombinant, comprising one or more heterologous nucleic acids. In certain embodiments, the cells are host cells, comprising one or more heterologous nucleic acids encoding one or more enzymes capable of catalysing the production of a compound of interest.

Suitable prokaryotic cells include, but are not limited, to any of a variety of gram-positive, gram-negative, or gram-variable bacteria. Examples include, but are not limited to, cells belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphlococcus, Strepromyces, Synnecoccus, and Zymomonas. Examples of prokaryotic strains include, but are not limited to: Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus. In a particular embodiment, the cell is an Escherichia coli cell.

Suitable archae cells include, but are not limited to, cells belonging to the genera: Aeropyrum, Archaeglobus, Halobacterium, Methanococcus, Methanobacterium, Pyrococcus, Sulfolobus, and Thermoplasma. Examples of archae strains include, but are not limited to: Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

Suitable eukaryotic cells include, but are not limited to, fungal cells, algal cells, insect cells, and plant cells. In some embodiments, yeasts useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the host is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorpha (now known as Pichia angusta). In some embodiments, the cell is a strain of the genus Candida, such as Candida hpolynca, Candida guilhermondn, Candida krusei, Candida pseudotropicalis, or Candida utilis.

In a particular embodiment, the cell is Saccharomyces cerevisiae. In some embodiments, the cell is a strain of Saccharomyces cerevisiae selected from the group consisting of Baker's yeast, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the cell is a strain of Saccharomyces cerevisiae selected from the group consisting of PE-2, CAT-1, VR-1, BG-1, CR-1, and SA-1. In a particular embodiment, the strain of Saccharomyces cerevisiae is PE-2. In another particular embodiment, the strain of Saccharomyces cerevisiae is CAT-1. In another particular embodiment, the strain of Saccharomyces cerevisiae is BG-1.

In some embodiments, the cell is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

In some embodiments, the cell is engineered to produce a C₅ isoprenoid. These compounds are derived from one isoprene unit and are also called hemiterpenes. An illustrative example of a hemiterpene is isoprene. In other embodiments, the isoprenoid is a C₁₀ isoprenoid. These compounds are derived from two isoprene units and are also called monoterpenes. Illustrative examples of monoterpenes are limonene, citranellol, geraniol, menthol, perillyl alcohol, linalool, thuj one, and myrcene. In other embodiments, the isoprenoid is a C₁₅ isoprenoid. These compounds are derived from three isoprene units and are also called sesquiterpenes. Illustrative examples of sesquiterpenes are periplanone B, gingkolide B, amorphadiene, artemisinin, artemisinic acid, valencene, nootkatone, epi-cedrol, epi-aristolochene, farnesol, gossypol, sanonin, periplanone, forskolin, and patchoulol (which is also known as patchouli alcohol). In other embodiments, the isoprenoid is a C₂₀ isoprenoid. These compounds are derived from four isoprene units and also called diterpenes. Illustrative examples of diterpenes are casbene, eleutherobin, paclitaxel, prostratin, pseudopterosin, and taxadiene. In yet other examples, the isoprenoid is a C₂₀₊ isoprenoid. These compounds are derived from more than four isoprene units and include: triterpenes (C₃₀ isoprenoid compounds derived from 6 isoprene units) such as arbrusideE, bruceantin, testosterone, progesterone, cortisone, digitoxin, and squalene; tetraterpenes (C₄₀ isoprenoid compounds derived from 8 isoprenoids) such as β-carotene; and polyterpenes (C₄₀₊ isoprenoid compounds derived from more than 8 isoprene units) such as polyisoprene. In some embodiments, the isoprenoid is selected from the group consisting of abietadiene, amorphadiene, carene, α-farnesene, β-farnesene, farnesol, geraniol, geranylgeraniol, isoprene, linalool, limonene, myrcene, nerolidol, ocimene, patchoulol, β-pinene, sabinene, γ-terpinene, terpinolene and valencene. Isoprenoid compounds also include, but are not limited to, carotenoids (such as lycopene, α- and β-carotene, α- and β-cryptoxanthin, bixin, zeaxanthin, astaxanthin, and lutein), steroid compounds, and compounds that are composed of isoprenoids modified by other chemical groups, such as mixed terpene-alkaloids, and coenzyme Q-10.

In some embodiments, the cell is engineered to produce a polyketide. In certain embodiments, the polyketide is selected from the group consisting of a polyketide macrolide, antibiotic, antifungal, cytostatic, anticholesterolemic, antiparasitic, a coccidiostatic, animal growth promoter and insecticide.

In some embodiments, the cell is engineered to produce a fatty acid.

Useful cells are described in WO 2015/095804, WO 2015/020649, and WO 2014/144135, the contents of which are hereby incorporated by reference in their entireties.

### 5.5 Recovery of Compounds

Once compound is produced by the cell culture, it may be recovered or isolated for subsequent use using any suitable separation and purification methods known in the art. In some embodiments, an organic phase comprising the compound is separated from the fermentation by centrifugation. In other embodiments, an organic phase comprising the compound separates from the fermentation spontaneously. In other embodiments, an organic phase comprising the isoprenoid is separated from the fermentation by adding a demulsifier and/or a nucleating agent into the fermentation reaction. Illustrative examples of demulsifiers include flocculants and coagulants. Illustrative examples of nucleating agents include droplets of the isoprenoid itself and organic solvents such as dodecane, isopropyl myristrate, and methyl oleate.

The compound produced in these cells may be present in the culture supernatant and/or associated with the cells. In embodiments where the compound is associated with the cells, the recovery of the isoprenoid may comprise a method of permeabilizing or lysing the cells. Alternatively or simultaneously, the compound in the culture medium can be recovered using a recovery process including, but not limited to, chromatography, extraction, solvent extraction, membrane separation, electrodialysis, reverse osmosis, distillation, chemical derivatization and crystallization.

In some embodiments, the compound is separated from other products that may be present in the organic phase. In some embodiments, separation is achieved using adsorption, distillation, gas-liquid extraction (stripping), liquid-liquid extraction (solvent extraction), ultrafiltration, and standard chromatographic techniques.

### 6. EXAMPLES

### 6.1 Example 1

This example provides a method demonstrating increased an exemplary method for determining the cell density (OD₆₀₀) of a yeast cell culture.

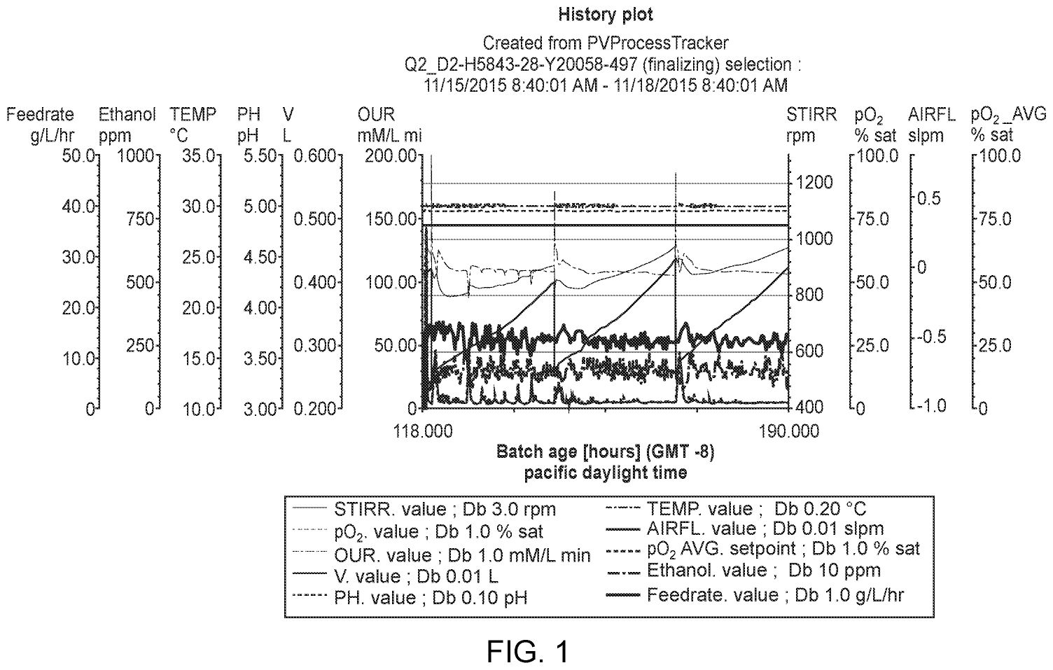

Two strains were evaluated in a continuous feeding process using target concentrations of 150, 300, and 600 ppm ethanol in the off-gas. Biomass was generated using typical pulse feeding processes. A production phase with continuous feeding started after approximately 48 hours. During production, the oxygen uptake rate (OUR) was controlled at 110 mmol/L/h and no dissolved oxygen spike checks were needed. A process trend of a continuous feeding process using ethanol off-gas is shown below in FIG. 1.

FIG. 1 provides an MFCS (BioPAT) trace of a continuous feeding process. The off-gas ethanol concentration (blue) was maintained at 150 ppm, by adjusting the feed rate (green). The OUR (red) was held constant by adjusting the agitation (dark green). The dissolved oxygen stayed near zero (black).

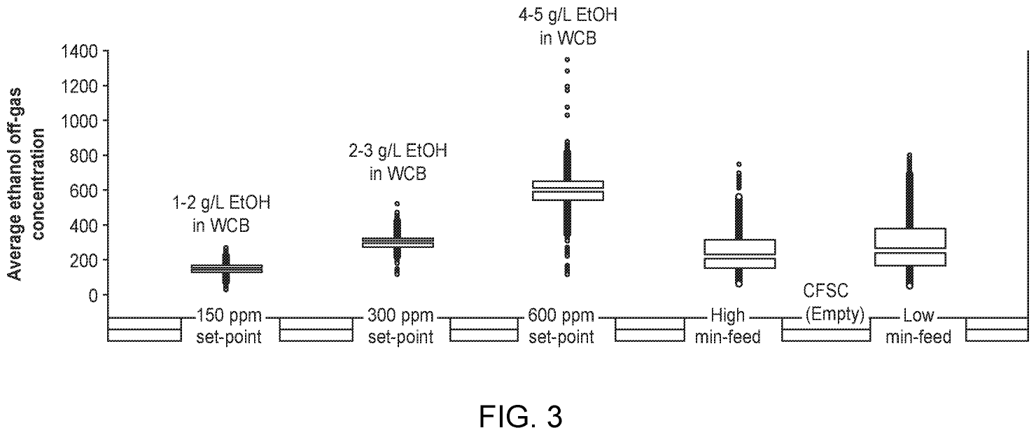

A 3-6 day interval was chosen in order to demonstrate this process. Both strains showed a trend of increasing productivity and no difference in yield with increasing off-gas ethanol concentration set-points as shown in FIG. 3. The figures shows that the productivity increases as the average ethanol concentration in the off-gas increases. The data also shows that the CFSC processes typically generate an average off-gas concentration of 220-320 ppm. Controlling the ethanol off-gas concentration at higher level by adjusting the feed-rate can give higher productivities for both strains.

The average ethanol off-gas concentrations for Strain A in the standard CFSC process and continuous feeding process is provided in FIG. 3. The CFSC process produces an average of 200-225 ppm ethanol in the off-gas and is much lower than the higher set-points for the continuous feeding experiments.

What is claimed is:

1. A method for providing sugar to a microbial culture comprising the steps of:
a. feeding sugar to a microbial culture growing in fermentation medium at an initial rate;
b. measuring ethanol concentration in off-gas from the fermentation medium;
c. when the ethanol concentration in the off-gas increases, decreasing the sugar feed rate; and
d. when the ethanol concentration in the off-gas decreases, increasing the sugar feed rate.

2. The method of claim 1, wherein sugar is fed to the fermentation medium continuously.

3. The method of claim 1, wherein the ethanol concentration in the off-gas is measured continuously.

4. The method of claim 1, wherein the sugar feed rate is maintained within ±25% of the initial feed rate.

5. The method of claim 1, wherein the sugar feed rate is maintained within ±15% of the initial feed rate.

6. The method of claim 1, wherein the sugar feed rate is maintained within ±10% of the initial feed rate.

7. The method of claim 1, wherein the feed rate is 1 to 25 g/L/hr.

8. The method of claim 1, wherein the oxygen uptake rate of the microbial culture is maintained.

9. The method of claim 1, wherein the oxygen uptake rate of the microbial culture is maintained by agitation.

10. The method of claim 1, wherein the oxygen uptake rate of the microbial culture is 1-150 mmol O₂/L/hr.

11. The method of claim 1, wherein the ethanol concentration in the off-gas is 50-750 ppm.

12. The method of claim 1, wherein the ethanol concentration in the off-gas is 100-200 ppm.

13. The method of claim 1, wherein the ethanol concentration in the off-gas is 200-300 ppm.

14. The method of claim 1, wherein the ethanol concentration in the off-gas is 250-350 ppm.

15. The method of claim 1, wherein the ethanol concentration in the off-gas is 550-650 ppm.

16. The method of claim 1, that is maintained for 1-10, 2-9, 3-7, or 4-6 days.

17. The method of claim 1, wherein the microbial culture is prokaryotic.

18. The method of claim 1, wherein the microbial culture is eukaryotic.

19. The method of claim 1, wherein the microbial culture is yeast.

20. The method of claim 1, wherein the microbial culture is S. cerevisiae.

21. The method of claim 1, wherein the microbial culture is recombinant.

22. The method of claim 1, wherein the microbial culture produces a water-immiscible compound.

23. The method of claim 1, wherein the microbial culture produces an isoprenoid, polyketide, or fatty acid.

24. The method of claim 1, wherein the microbial culture produces a sesquiterpene.
